(19) United States
(12) Patent Application Publication
(10) Pub. No.: US 2011/0087187 A1
(43) Pub. Date: Apr. 14, 2011

(54) HYDROPHOBIC DEAERATION MEMBRANE

(75) Inventor: Christof Beck, Blitz (DE)

(73) Assignee: GAMBRO LUNDIA AB, Lund (SE)

(21) Appl. No.: 12/937,928

(22) PCT Filed: Apr. 29, 2009
(86) PCT No.: PCT/EP09/03110

§ 371 Date: Dec. 17, 2010

(30) Foreign Application Priority Data
Apr. 30, 2008 (EP) 08008248.0

(51) Int. Cl. A61M 31/00; B01D 19/04; B01D 71/70; B01D 71/36; B01D 67/00; B05D 5/00

(52) U.S. Cl. 604/500; 96/6; 95/46; 427/244

(57) ABSTRACT

An optimized deaeration membrane has a biocompatible coating composition. Methods for preparing the membrane and the use of the membrane in medical devices for separating air from liquid that are administered to a living subject, e. g., blood processing devices used in dialysis and the like, are also described.

### BACKGROUND OF THE INVENTION

This invention relates to an optimized deaeration membrane having a biocompatible coating composition which is to be applied to blood processing devices such as for dialysis or the like. In a specific embodiment, the invention relates to a hydrophobic deaeration membrane with a biocompatible coating comprising a polysiloxane and silicon dioxide particles, methods for preparing the membrane and the use of the membrane in medical devices for separating air from liquids that are administered to a living subject.

The medical treatment of body liquids of living subjects generally involves medical devices, such as degassing devices, for removing or separating air from said liquids before there are administered or transferred back to the individual. During blood processing, air is often mixed with the blood necessitating the removal of air bubbles or the “defoaming” of the blood before returning it to the patient. Defoaming is typically accomplished by providing a large surface area which is covered by a so-called defoaming or anti-foaming agent. The surface area is often composed of a synthetic material, such as polyurethane foam, polypropylene mesh, polyvinylchloride strips, or stainless steel wool. Various defoaming agents that prevent or dissipate foam are known to those skilled in the art.

Such degassing devices are used in various treatments of blood, such as blood autotransfusion and cell separation during an operation, such as, for example, cardio-pulmonary bypass procedures, but also especially in hemodialysis, hemofiltration, haemodiafiltration or plasmapheresis applications. In all these treatments, blood is withdrawn from a patient, passed through a filter, such as a dialyzer, and returned to the patient. As blood is returned to the patient, it is treated for the removal of particles and especially for the removal of gas bubbles.

Gas bubbles, even if they are very small, can cause serious damage to body functions by causing air embolism. Air embolism occurs when bubbles of air become trapped in the circulating blood. An embolus in an artery is travelling in a system of blood vessels that are gradually getting smaller. At some point a small artery will be blocked and the blood supply to some area of the body is cut off. The effects of the blockage will depend on the part of the body to which the artery supplies blood. If, for example, the embolism prevents blood supply to the brain, tissues will be starved of oxygen, causing them to die. If this happens, it can cause permanent brain damage. If the embolus is in a vein, the blood vessel system widens along the direction of the blood flow, so a small embolus may not do much harm until it passes through the heart, after which it enters an artery.

Present filters or membranes that have been used to remove gas from liquids have often included hydrophobic or water-repellent membranes. Such hydrophobic membranes permit gas to pass but prevent the passage of a liquid.

U.S. Pat. No. 5,541,167 A describes a composition for coating medical blood contacting surfaces which comprises a mixture of an anticoagulant and a defoaming agent. The coating composition is applied by either dipping the device into a solution containing the mixture or by spraying the mixture onto the surface. In a preferred embodiment, the anticoagulant is a quaternary ammonium complex of heparin and the anti-foaming agent is a mixture of polydimethylsiloxane and silicon dioxide, such as SIMETHICONE or the compound marketed by Dow Corning under the trade name ANTIFOAM A®. In Example 2 of U.S. Pat. No. 5,541,167 A, a polyurethane defoamer is dip-coated in 5% (w/v) of ANTIFOAM A®.

U.S. Pat. No. 6,506,340 B1 discloses medical devices comprising hydrophobic blood-contact surfaces durably coated with a non-toxic, biocompatible surface-active defoaming agent. The defoaming agent is selected from polyethers consisting essentially of block copolymers of propylene oxide and ethyl-ene oxide. Silicone-based surfactants are used as comparative defoaming agents in the examples of U.S. Pat. No. 6,506,340 B1.

U.S. Pat. No. 3,631,654 A describes filters used in devices for venting gases, wherein a portion of the filter is wetted by liquids and another portion of the same filter is liquid repellent. U.S. Pat. No. 3,631,654 discloses that hydrophilic membranes, e.g. a membrane made from crocidolite-type asbestos fibers and an amyl acetate binder, may be rendered hydrophobic by treatment with a 5 percent solution of silicone resin in perchloroethylene.

U.S. Pat. No. 6,267,926 B1 discloses an apparatus for removing entrained gases from a liquid that comprises a hydrophobic microporous membrane material through which the gases are withdrawn from the liquid by the application of a negative pressure. The membrane is preferably made from a material selected from the group consisting of polypropylene, polyethylene, polyurethane, polymethylpentene, and polytetra-fluoroethylene.

GB 2 277 886 A and U.S. Pat. No. 4,572,724 A describe a filter having provisions for degassing blood which comprises an upstream sponge-structure degassing filter element and vent outlets bridged by a liquophobic PTFE membrane which allows gas to pass through. The sponge-structure degassing filter element may be treated with an antifoaming agent, for example, a compound of silicone and silica, such as ANTIFOAM A®.

U.S. Pat. No. 4,190,426 A discloses venting filters comprising vent opening means covered by a liquid-repellent filter made from polytetrafluoroethylene.

U.S. Pat. No. 4,210,697 A describes a process for preparing hydrophobic porous fibrous sheet material for use as a filter, wherein a porous fibrous substrate, e.g. a woven cloth of glass or mineral wool fiber, is impregnated with an aqueous dispersion comprising polytetrafluoroethylene and silicone resin prepolymer, e.g. reactive polydimethylsiloxane.

U.S. Pat. No. 4,004,587 A discloses a filter comprising first and second filter members in parallel flow position, wherein the first filter member is hydrophilic and the second filter member is hydrophobic. The hydrophobic filter membrane may be a copolymer of polyvinyl chloride and acrylonitrile placed on a nylon fabric substrate and treated with an organosilicon compound to render it hydrophobic, or it may be made from porous polytetrafluoroethylene.

U.S. Pat. No. 5,286,279 A discloses a gas permeable material having continuous pores through it, made by coating the interiors of the pores of a membrane material selected from the class consisting of porous polytetrafluoroethylene, porous polyamides, porous polyesters, porous polycarbonates, and porous polyurethanes, with the reaction product of a diisocyanate and a perfluoroalkyl alcohol. The resulting membranes are reported to be both hydrophobic and oleophobic.

U.S. Pat. No. 5,123,937 A discloses a stratified membrane structure for use in deaerating modules, formed by laminating a solid gas-permeable layer to a fibrillated porous resin film. For instance, a polytetrafluoroethylene film is expanded and a solid layer consisting of a silicone or a fluorosilicone having a film thickness ranging from 1 to 150 microns is coated or laminated on the resulting film.

EP 1 019 238 B1 describes layered membrane structures with a stratified pore structure produced by calendering two or more extrudate ribbons made from expanded PTFE or expanded interpenetrating polymer networks of PTFE and silicone. The membranes are said to be suitable for medical applications where a pore size gradient is desired.

### BRIEF DESCRIPTION OF THE FIGURES

FIG. 1 shows an electron microscopy photograph (x 1200) of a membrane showing uniform distribution of silicon dioxide particles. Circle A shows a region of the membrane with PDMS, Circle B a silicon dioxide particle, Circle C a part of the PTFE membrane with pores.

FIG. 2A depicts how the outer, middle and inner region of a membrane can be defined according to the invention. FIG. 2B shows where pictures are taken for the assessment of the particle distribution by electron microscopy.

FIG. 3 shows examples of a silicon dioxide particle distribution with a particle density above the optimal range, i.e. above 32000 particles per mm² (A) and below the optimal range, i.e. below 22000 particles per mm² (B), respectively.

FIG. 4 shows a membrane having an optimal coating with regard to silicon dioxide particle distribution, polysiloxane distribution and number and size of freely accessible membrane areas. The figures shown are electron microscopy images of the membrane, showing the middle (4A), inner (4B) and outer (4C) region of the membrane. The white arrows indicate 50 μm.

FIG. 5 shows examples of the deaeration profiles obtained from a deaeration device using membranes having coatings that are rated as good (“A”), “inhomogeneous (”C″) and unacceptable (“E”). The membrane having a good coating produces a deaeration profile depicted as “−”; 100% deaeration is reached within less than 30 seconds. The membrane having an inhomogeneous coating and rated “C” produces a deaeration profile depicted as “. . . ”; 100% deaeration is reached after more than 3 minutes. The membrane having an unacceptable coating and rated “E” produces a deaeration profile depicted as “− −”; less than 70% of the air within the system is vented through the membrane.

### DESCRIPTION OF THE INVENTION

According to one aspect of the invention, a deaeration membrane having a biocompatible coating composition is provided which removes air bubbles and reduces blood trauma during extracorporeal circulation by allowing said air bubbles to pass through the membrane.

The deaeration membrane comprises a flexible, porous, polymeric material having passageways, or continuous pores, through the material. The material comprises porous polytetrafluoroethylene (PTFE). The porous PTFE material may be a sheet having a thickness of from 0.15 to 0.30 mm, or even from 0.20 to 0.25 mm.

An example of a suitable PTFE membrane is a membrane made of expanded PTFE having a pore size of 0.2 μm, available from W. L. Gore & Associates, Inc. under the trade name GORE™ MMT-323.

The deaeration membrane further comprises a coating comprising a defoaming agent. Typical defoaming agents are comprised of both active compounds and carriers. Occasionally, the agents will also include a spreading agent. Typical active compounds include fatty acid amides, higher molecular weight polyglycols, fatty acid esters, fatty acid ester amides, polyalkylene glycols, organophosphates, metallic soaps of fatty acids, silicone oils, hydrophobic silica, organic polymers, saturated and unsaturated fatty acids, and higher alcohols. Typical carriers include paraffinic, napthenic, aromatic, chlorinated, or oxygenated organic solvents. Those skilled in the art will be able to determine the appropriate composition of the defoaming agent depending upon the application. Preferred defoaming agents to apply to the deaeration membrane of the invention are polysiloxanes, in particular polydimethylsiloxane (PDMS). A mixture of polydimethylsiloxane and silicon dioxide is used in one embodiment. However, instead of PDMS, which is readily available and can easily be applied, other silicone resin prepolymers can be used, including polymethylethylsiloxane, polydiethylsiloxane, polydipropylsiloxane, polydihexylsiloxane, polydiphenylsiloxane, polyphenylmethylsiloxane, polydicyclohexylsiloxane, polydicyclopentylsiloxane, polymethylcyclopentylsiloxane, polycyclohexylsiloxane, polydicycloheptylsiloxane, and polydicyclobutylsiloxane.

In a particular embodiment, the defoaming agent is Simethicone, USP (CAS: 8050-81-5) or a composition comprising >60 wt. % polydimethylsiloxane (CAS:63148-62-9), 7-13 wt. % methylated silica (CAS: 67762-90-7), 3-7 wt. % octamethylcyclotetrasiloxane (CAS:556-67-2), 3-7 wt. % decamethylcyclopentasiloxane (CAS: 5541-02-6), 1-5 wt. % dimethylcyclosiloxanes and 1-5 wt. % dodecamethylcyclohexasiloxane (CAS:540-97-6), which can be purchased from Dow Corning Corp. under the trade name Antifoam A®.

The PDMS, for example, acts as a surfactant and reduces the surface tension of the air bubbles to merge to larger bubbles in blood when they come into contact with the membrane surface. This allows smaller air bubbles to merge to larger bubbles, which have a higher probability of being vented through the membrane due to their larger surface area. The silicon dioxide particles act as a mechanical rupture in order to break up the thin protein film that tends to form around air bubbles.

The silicon dioxide particles usually have a particle size in the range of from 0.1 to 50 μm, for example 1 to 20 μm, 0.1 to 5 μm or 1 to 15 μm. The particles can be agglomerates of smaller primary particles having a particle size in the range of from 10 to 500 nm, for instance 20 to 200 nm, or 10 to 50 nm, or 10 to 30 nm.

In one embodiment, the membrane comprises a PTFE membrane coated with a defined amount of a defoaming agent. The amount of the defoaming agent (for example, Antifoam A®), present per face of the membrane may range from 4 μg/mm² to 15 μg/mm², for instance 4.25 μg/mm² to 10 μg/mm², or even from 4.25 μg/mm² to 7.10 μg/mm². In a particular embodiment, only one face of the membrane is coated.

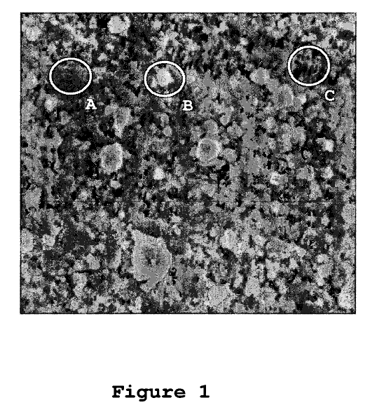

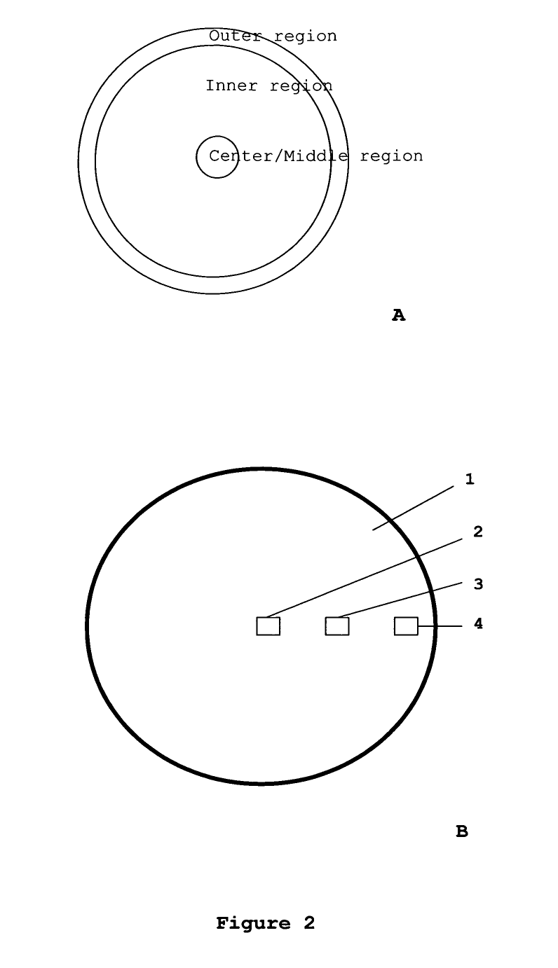

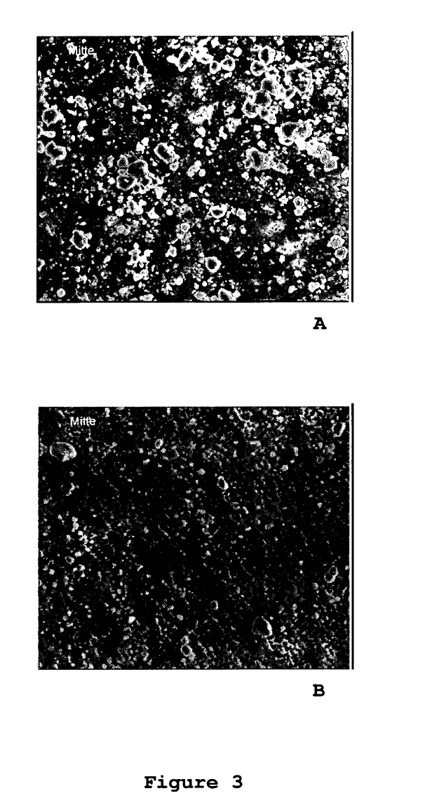

In a certain embodiment of the invention, the membrane exhibits an even or uniform distribution of silicon dioxide (silica) particles throughout the entire coated surface of the membrane, including the inner, middle and outer regions of the membrane (see FIGS. 1 and 2). The number of silica particles (FIG. 1B) preferably is in the range of from 22,000 to 32,000 particles per mm², or even from 25,000 to 30,000 particles per mm². A particle concentration of less than about 22,000 (FIG. 3B) or more than 32,000 (FIG. 3A) particles per mm² in any part of the membrane will result in a decrease in degassing efficiency.

In another embodiment of the invention, the membrane exhibits a patterned distribution of silicon dioxide particles, comprising a regular pattern of areas covered with silicon dioxide particles and areas free of silicon dioxide particles. Such a pattern can be generated by roll coating the membrane using an anilox roll, a gravure roll or screen-printing with a mesh. In this embodiment the particle concentration in the areas covered with silicon dioxide particles can be higher than 32,000 particles per mm², for instance up to 50,000 particles per mm², or even 70,000 particles per mm², provided that the average particle concentration on the coated surface of the membrane does not exceed 44,000 particles per mm², for instance is not higher than 40,000 particles per mm². In a particular embodiment, the proportion of the areas free of silicon dioxide particles is 10 to 30 percent of the total membrane surface, for instance 20 to 25 percent.

The deaeration membrane may be in sheet form and the coating coats at least a portion of the interior of the pores of the PTFE membrane but does not fully block the pores (see FIG. 1, especially FIG. 1C). Thus, the gas permeability of the membrane material remains unhampered.

The deaeration membrane may have a pore size that is sufficiently small to keep bacteria from passing through the membrane. A desirable mean average pore size is 0.2 μm or smaller.

The deaeration membrane of the present application is optimized for direct blood contact. Prior art hydrophobic membranes, when brought into direct blood contact, suffer from (a) protein adsorption from the blood onto the membrane which causes clogging of the membrane pores, and (b) gas bubbles remaining on the deaeration membrane surface without being vented, as the surface tension of the gas bubbles in blood cannot be overcome by a PTFE surface upon direct contact, both processes resulting in reduced deaeration performance. The deaeration membrane of the present application provides the advantage of reduced clogging of the membrane and faster venting of any gas bubbles, independent of their size, through the membrane of the invention than through a membrane without the defoaming coating. Since air bubble accumulation under the deaeration membrane is thus reduced, the probability that air bubbles can pass the deaeration device downstream is lower. Accordingly, the air trapping or degassing efficacy of such a membrane and any device using such a membrane will be higher. Moreover, the deaeration performance of the membrane or any device equipped with such a membrane will be stable over several hours of usage, e.g. during a dialysis treatment.

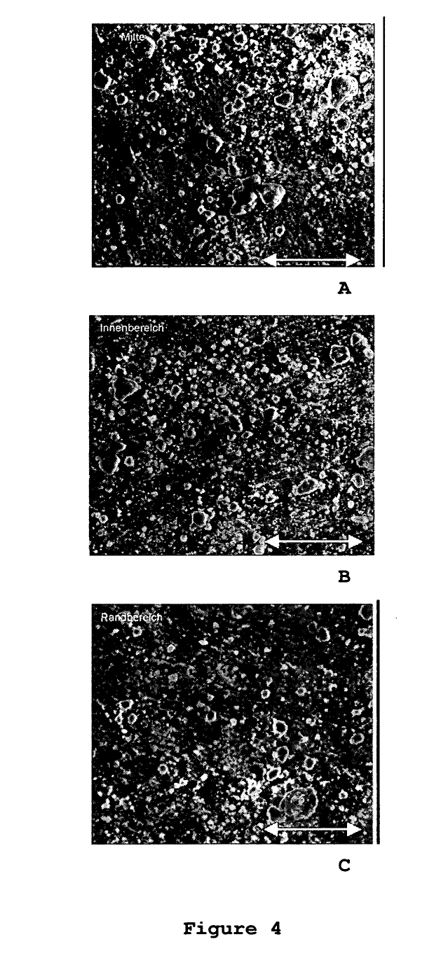

The present application also provides for a method of preparing the deaeration membrane. The method comprises coating a membrane comprising porous polytetrafluoroethylene with a defoaming agent, e.g. a defoaming agent comprising a polysiloxane and silica particles. In a specific embodiment, the invention provides a method for coating a PTFE membrane, which results in uniform particle distribution in the inner (FIG. 4B), middle (FIG. 4A) and outer (FIG. 4C) regions, respectively, of a given membrane (FIG. 4).

In a particular embodiment, the coating on the PTFE membrane is produced by dissolving the defoaming agent in a solvent and subsequently dip-coating the membrane in the solution or spray-coating the solution onto the membrane. For obtaining a uniform coating, it is an option to spray-coat the solution on the membrane. The person skilled in the art is familiar with methods of spray-coating a solution onto a membrane. In a particular embodiment, a two-substance nozzle employing air, steam or other inert gases to atomize liquid is used for spray-coating. The pressure of the atomizing gas may be greater than 0.3 bar to achieve a large specific surface and uniform distribution. In one embodiment, the nozzle orifice ranges from 0.3 to 1 mm. In a particular embodiment, the nozzle produces a full circular cone with an aperture of from 10° to 40°. The mass flow of the solution, the distance between the nozzle and the membrane to be coated, and the lateral relative velocity of the membrane and the nozzle preferably are selected to produce a coating comprising from 4.25 μg/mm² to 10 μg/mm², or even from 4.25 μg/mm² to 7.10 μg/mm² of defoaming agent (after removal of solvent present in the solution). In an exemplary embodiment, a nozzle is used which sprays the solution with a mass flow of about 5-10 ml/min onto the membranes which are transported past the nozzle at a velocity of about 175-225 cm/min.

In another embodiment, the coating on the PTFE membrane is produced by roll coating the solution onto the membrane. In a particular embodiment, roll coating is performed using an anilox roll. Examples of suitable roll coating techniques are gravure coating and reverse roll coating. Coating parameters are preferably set to produce a coating comprising from 4 μg/mm² to 15 μg/mm², for example 4.25 to 10 μg/mm² or even from 4.25 μg/mm² to 7.10 μg/mm² of defoaming agent (after removal of solvent present in the solution).

The defoaming agent can be dissolved in an appropriate solvent before using it for coating a membrane. Such a solution may, for example, contain the defoaming agent in a concentration of from 0.1 wt.-% to 20 wt.-%, e.g. from 1 wt.-% to 10 wt.-%, or even from 3 wt.-% to 8 wt.-%. In case the solution is to be used for roll coating, higher concentrations of the defoaming agent are generally suitable. For example, the solution may contain the defoaming agent in a concentration of from 20 wt.-% to 70 wt.-%, for instance 25 to 50 wt.-%.

The solvent for the defoaming agent used is not particularly limited, if the polysiloxane compound, the silicon dioxide particles and the solvent are appropriately mixed, and if no significant difficulties are caused by phase separation. However, it is proper to use aliphatic hydrocarbons such as n-pentane, i-pentane, n-hexane, i-hexane, 2,2,4-trimethylpentane, cyclohexane, methylcyclohexane, etc.; aromatic hydrocarbons such as benzene, toluene, xylene, trimethylbenzene, ethylbenzene, methyl ethyl benzene, etc.; alcohols such as methanol, ethanol, n-propanol, propanol, n-butanol, i-butanol, sec-butanol, t-butanol, 4-methyl-2-pentanol, cyclohexanol, methylcyclohexanol, glycerol; ketones such as methyl ethyl ketone, methyl isobutyl ketone, diethyl ketone, methyl n-propyl ketone, methyl n-butyl ketone, cyclohexanone, methylcyclohexanone, acety-lacetone, etc.; ethers such as tetrahydrofuran, 2-methyltetrahydrofuran, ethyl ether, n-propyl ether, isopropyl ether, diglyme, dioxane, dimethyldioxane, ethylene glycol monomethyl ether, ethylene glycol dimethyl ether, ethylene glycol diethyl ether, propylene glycol monomethyl ether, propylene glycol dimethyl ether, etc.; esters such as diethyl carbonate, methyl acetate, ethyl acetate, ethyl lactate, ethylene glycol monomethyl ether acetate, propylene glycol monomethyl ether acetate, ethylene glycol diacetate, etc.; and amides such as N-methylpyrrolidone, formamide, N-methyl formamide, N-ethyl formamide, N,N-dimethyl acetamide, N,N-dimethyl acetamide, etc. In a particular embodiment, aliphatic hydrocarbons such as n-pentane, pentane, n-hexane, i-hexane, 2,2,4-trimethylpentane, cyclohexane, methylcyclohexane, etc. are used. In another particular embodiment, n-hexane is used as a solvent.

In one embodiment of the spray-coating process, the solution of the defoaming agent is cooled down before application in order to avoid evaporation of the solvent during the spray-coating process. For instance, the solution used in the spray-coating process is cooled down to a temperature of from 0 to 15° C., e.g. 0 to 10° C., or even 0 to 5° C.

The coated membrane is then dried, e.g. at room temperature, for about 30 minutes to two hours, e.g. for about one hour. However, it is also possible to dry the membranes at elevated temperatures of up to 200° C. to shorten the time that is needed for drying. In case the amount of coating (in weight per mm²) resulting from the first coating procedure is below the desired range, the same membrane can be subjected to a second coating procedure as described above.

A further subject of the present application is the use of the deaeration membrane of the invention for removing entrained gases from a liquid. In one embodiment of the invention, the liquid comprises protein. Protein-comprising liquids have an increased tendency to form foams. In a particular embodiment, the liquid is blood. In one embodiment of the invention, the liquid, from which entrained gases have been removed, is administered to a living subject. Examples are hemodialysis and extracorporeal circulation. The membrane of the invention can be used in a degassing device. An advantage of the membrane of the present application is that it can be in direct contact with the liquid during use.

### EXAMPLES

### Example 1

Spray coating a hydrophobic PTFE membrane with Antifoam A® 30 g of Antifoam A® were dissolved in 570 g of hexane and the solution was stirred for 5 minutes at room temperature, followed by cooling on ice for 10 minutes. The solution was then sprayed on a row of membranes by means of a nozzle (Two-substance nozzle Type 970/0, Orifice 0.3 mm, spray pattern: full circular cone of 10° to 40° produced by Düsen-Schlick GmbH, 96253 Untersiemau/Coburg, Germany, air pressure 0.4 bar). The device for spraying was cooled where possible to avoid an early evaporation of the solvent. The membranes passed the nozzle on a slide with a velocity of 200 cm per minute. The membranes were then dried at room temperature for one hour.

The mass gain of each membrane was determined and the quality of the coating of each membrane in the outer, inner and middle region of the membrane was analyzed by electron microscopy (FIG. 2). Special attention was given to the accessibility of the membrane pores, silicon dioxide particle distribution and the distribution of the PDMS (FIGS. 1 and 4). The coating quality was rated as follows: a first rating was given for the total amount of coating substance on the membrane (707 mm²) in mg. A value of “100” was given for an amount of between 4 and 5 mg per membrane, “90” for an amount of between 3 and 4 mg and between 5 and 6 mg, respectively, “80” for an amount of between 2 and 3 mg and between 6 and 7 mg, respectively, and “0” for an amount of below 2 mg and above 7 mg. Further ratings were based on the results of the electron microscopy, ranging from a value of “100” for a uniform distribution of silicon dioxide particles, PDMS and freely accessible membrane areas, to values of “0” for silicon dioxide particle concentrations below 22000 particles per mm² or above 32000 particles per mm², or a complete lack of PDMS or freely accessible membrane areas. A rating was evaluated for each of the middle, outer and inner regions of the membrane. An average value was calculated from the four ratings and the membrane was assigned to one of five classes “A” to “E”, with “A” corresponding to an average value of >90 to 100, “B” corresponding to an average value of >80 to 90, “C” corresponding an average value of >70 to 80, “D” corresponding to an average value of >60 to 70, and “E” corresponding to an average value of 60 or less.

Table I shows the results of such rating for three membranes, indicating the mass gain together with a first mass rating and the ratings based on the electron microscopy analysis (Rating EM) as described above, for the middle, inner and outer regions of the membrane as well as the overall ratings for each membrane.

TABLE I

| Membrane | Mass Gain | Rating | Rating EM | Rating EM | Rating EM | Rating | Rating | Rating | Rating | Rating |
|---|---|---|---|---|---|---|---|---|---|---|
| No. | [mg] | Mass | Middle | Inner | Outer | “A” | “B” | “C” | “D” | “E” |
| 1 | 2.9 | 80 | 30 | 30 | 50 | | | | | x |
| 2 | 3.1 | 90 | 70 | 60 | 70 | | | x | | |
| 3 | 4.2 | 100 | 85 | 90 | 85 | x | | | | |

### Determination of the Degassing Efficiency of a Membrane

The degassing efficiency was tested in a clinical setting for haemodialysis. The membranes were used within a degassing device that was located either on the venous or arterial side of the dialyser. The dialysis system comprised a standard dialysis setup including an AK 200 Ultra dialysis machine and a Polyflux® 170 H dialyser.

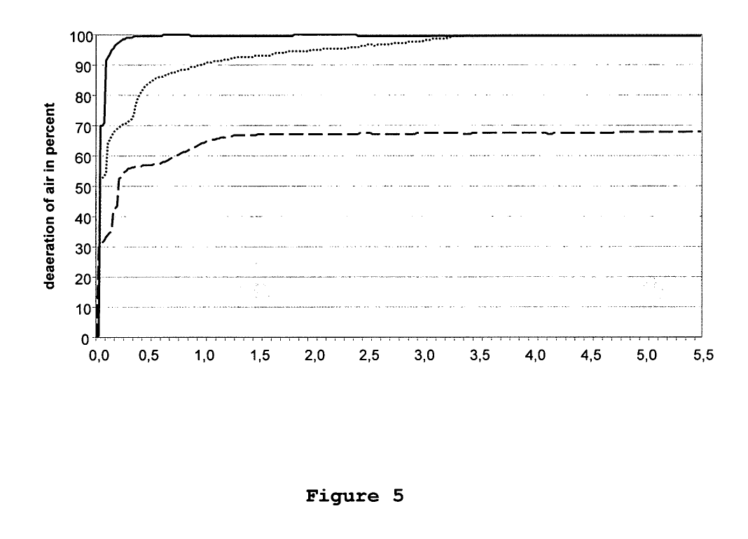

The degassing or deaeration efficiency was determined by injecting air into the system and measuring the amount of air leaving the system and the time period required for deaeration. The deaeration efficiency is plotted as deaeration of air in percent over time. FIG. 5 shows the results obtained for three different membranes. The membrane rated “A” produced the deaeration profile depicted as “−” and 100% deaeration was achieved within less than 30 seconds. The membrane rated “C” and had an inhomogeneous coating produced the deaeration profile depicted as “. . . ”. In this case, 100% deaeration was achieved only after more than 3 minutes. The membrane rated “E” and having an unacceptable coating (see FIG. 3) produced the deaeration profile depicted as “− −”. In this case, less than 70% of the air within the system was vented through the membrane.

### Example 2

Roll coating a hydrophobic PTFE membrane with Antifoam A® Two coating solutions comprising 25 wt.-% Antifoam A® in hexane and 50 wt.-% Antifoam A® in hexane, respectively, were prepared. Membranes were coated with the solutions using an anilox roll having 25 cells per mm, each cell having a depth of 0.142 mm. The theoretical volume of the cells was 43.49 ml/m². The coated membranes were dried at 200° C. in a circulating air oven.

Silicon dioxide particle concentrations on the coated membranes were evaluated by SEM. For the membrane coated with the 25 wt.-% solution, a concentration of 25,000 particles per mm² was found in the areas corresponding to the cells of the anilox roll, while for the membrane coated with the 50 wt.-% solution, a concentration of 50,000 particles per mm² was determined.

The degassing efficiency of the coated membranes was tested as described above. With both membranes, 100% deaeration was achieved within one minute.

As various changes could be made without departing from the scope of the present invention, it is intended that all matter contained in the above description or shown in the accompanying drawings shall be interpreted as illustrative and not limiting.

1. A deaeration membrane comprising porous polytetrafluoroethylene in sheet form coated with a defoaming agent comprising a polysiloxane and silicon dioxide particles, wherein the amount of coating is from 4 μg per mm² to 15 μg per mm² of the membrane and the silicon dioxide particles are present in an average concentration of from 22,000 particles per mm² to 44,000 particles per mm² of the coated deaeration membrane.

2. The membrane of claim 1, wherein the porous polytetrafluoroethylene sheet has a thickness of 0.15 mm to 0.30 mm.

3. The membrane of claim 1 wherein only one face of the polytetrafluoroethylene sheet is coated with the defoaming agent.

4. The membrane of claim 1 wherein the polysiloxane comprises polydimethylsiloxane.

5. A method for producing a deaeration membrane comprising porous polytetrafluoroethylene in sheet form coated with a defoaming agent comprising a polysiloxane and silicon dioxide particles, wherein the amount of coating is from 4 μg per mm² to 15 μg per mm² of the membrane and the silicon dioxide particles are present in an average concentration of from 22,000 particles per mm² to 44,000 particles per mm² of the coated deaeration membrane, the method comprising spray-coating a sheet of porous polytetrafluoroethylene with a solution comprising 0.1 wt. % to 20 wt. % of a defoaming agent comprising a polysiloxane and silicon dioxide particles.

6. The method of claim 5 further comprising cooling the solution to a temperature of from 0° C. to 15° C. before it is used for spray-coating the sheet of porous polytetrafluoroethylene.

7. A method for producing the a deaeration membrane comprising porous polytetrafluoroethylene in sheet form coated with a defoaming agent comprising a polysiloxane and silicon dioxide particles, wherein the amount of coating is from 4 μg per mm² to 15 μg per mm² of the membrane and the silicon dioxide particles are present in an average concentration of from 22,000 particles per mm² to 44,000 particles per mm² of the coated deaeration membrane, the method comprising roll coating a sheet of porous polytetrafluoroethylene with a solution comprising 20 wt. % to 70 wt. % of a defoaming agent comprising a polysiloxane and silicon dioxide particles.

8. The method of claim 7, wherein roll coating a sheet of porous polytetrafluoroethylene comprises roll coating the sheet of porous polytetrafluoroethylene with an anilox roll.

9. The method of claim 8 wherein roll coating a sheet of porous polytetrafluoroethylene with a solution comprising 20 wt. % to 70 wt. % of a defoaming agent comprising a polysiloxane and silicon dioxide particles comprises roll coating the sheet of porous polytetrafluoroethylene with a solution comprising 20 wt. % to 70 wt. % of a defoaming agent comprising polydimethylsiloxane and silicon dioxide particles.

10. A method for removing entrained gases from a liquid comprising passing the liquid containing the entrained gases through a deaeration membrane comprising porous polytetrafluoroethylene in sheet form coated with a defoaming agent comprising a polysiloxane and silicon dioxide particles, wherein the amount of coating is from 4 μg per mm² to 15 μg per mm² of the membrane and the silicon dioxide particles are present in an average concentration of from 22,000 particles per mm² to 44,000 particles per mm² of the coated deaeration membrane.

11. The method according to claim 10 for removing entrained gases from a liquid containing protein.

12. The method according to claim 11 for removing entrained gases from blood.

13. The method according to claims 10 further comprising administering the liquid to a living subject.

14. The method according to claims 10 comprising placing the membrane is in direct contact with the liquid.

15. The method according to claims 10 further comprising placing the membrane in a deaeration device.

16. The membrane of claim 2 wherein only one face of the polytetrafluoroethylene sheet is coated with the defoaming agent.

17. The membrane of claim 2 wherein the polysiloxane comprises polydimethylsiloxane.

18. The membrane of claim 3 wherein the polysiloxane comprises polydimethylsiloxane.

19. The membrane of claim 16 wherein the polysiloxane comprises polydimethylsiloxane.

20. The method of claim 7 wherein roll coating a sheet of porous polytetrafluoroethylene with a solution comprising 20 wt. % to 70 wt. % of a defoaming agent comprising a polysiloxane and silicon dioxide particles comprises roll coating the sheet of porous polytetrafluoroethylene with a solution comprising 20 wt. % to 70 wt. % of a defoaming agent comprising polydimethylsiloxane and silicon dioxide particles.
